(19) United States
(12) Patent Application Publication
(10) Pub. No.: US 2003/0144621 A1
(43) Pub. Date: Jul. 31, 2003

(54) Portable lumbar stabilization and extremities rehabilitation muscle feedback kit

(76) Inventors: Paul Osterbye, Tallahassee, FL (US); Suzanne Ambrose, Tallahassee, FL (US)

Correspondence Address: Carnes, Cona & Dixon, Innovation park, 1673 West Paul Dirac Drive, Tallahassee, FL, 32310-3763, US

(21) Appl. No.: 10/338,135

(22) Filed: Jan. 8, 2003

Related U.S. Application Data
(60) Provisional application No. 60/346,831, filed on Jan. 10, 2002.

(51) Int. Cl. A61F 5/00

(52) U.S. Cl. 602/13

(57) ABSTRACT

The present invention is a novel and unique portable and adaptable lumbar stabilization and upper and lower extremities rehabilitation muscle feedback device. The inventive devices and methods allow healthcare professionals as well ass patient to accurately assess the patient during the use of “neutral spine” exercises. A bladder is retained against a portion of the body and pressurized. The bladder pressure is monitored to encourage the user to maintain controlled contraction of the target muscles, thereby increasing proprioception and neuromuscular control.

### BACKGROUND OF THE INVENTION

1. Field of the Invention

The present invention relates generally to a portable and adaptable lumbar stabilization and upper and lower extremities rehabilitation muscle feedback kit to be used by the healthcare professional or the patient to measure changes in forces exerted upon the lumbar spine and the upper and lower extremities and more particularly to a portable and adaptable kinemometer stabilization program kit which is an excellent resource for proprioceptive retraining of the lumbar spine and postular education, as well as achievement of proprioception and neuromuscular control in the rehabilitation of upper and lower extremities.

2. Description of the Prior Art

Utilization of the lumbar spine stabilizers is the basis for spine stabilization exercises. In recent times, there has been an increase in the use of spine stabilization exercises for the rehabilitation and prevention of injury to the lumbar spine. The use of “neutral spine” exercises and activities of abdominal bracing are becoming widely accepted as effective treatment of lumbar spine conditions. These exercises can, however, be performed without actually engaging the muscles which stabilize the lumbar spine. Hence, if lumbar spine stabilization exercises are indicated, it is critical that the lumbar spine stabilizing muscles are actually engaged in exercise.

It is evidenced in the prior art that numerous attempts have been made to address the problems associated with the current rehabilitation and prevention techniques and devices used to treat injury to the lumbar spine and other areas. For example, U.S. Pat. No. 5,338,276 issued to Jull discloses an exercise monitoring device which provides for a process of self monitoring of physiotherapy or physical therapy exercise which involves muscular movement including stretching of muscles. Though successful in a process for self-monitoring, the Jull devices suffer some shortcomings. One such shortcoming is that Jull does not provide for a portable and adaptable kit, which largely restricts its versatility and usability. In addition, the Jull devices are limited in the number of muscle groups it serves.

Another example can be seen in U.S. Pat. No. 5,980,560 issued to Chang discloses an air injection type stomach band for distracting the lumbar vertebrae of disc patients. A plurality of air expansion tubes comprising the stomach band are connected perpendicularly to an air injection tube at equal distant intervals.

Another example is seen in U.S. Pat. No. 5,450,858 issued to Zablotsky et al wherein disclosed is an air inflatable belt worn by a person for support in the lumbar and sacral region of the body which has an inner wall, which faces the body of the person, comprised of one or more permanent magnet arrangements producing magnetic fields directed toward the persons body.

Yet another example can be seen in U.S. Pat. No. 5,195,948 issued to Hill et al which discloses a back support device comprising a belt structure designed to fit substantially around the waist of the user. An inflatable air bladder is attached inside the belt structure in order to be positioned adjacent to the lower back.

Still another example is seen in U.S. Pat. No. 4, 703,750 issued to Sebastian et al wherein disclosed is a therapeutic appliance for application to the lumbar spine and follows the contours of the iliac crest and overlies sacrum and the sacrial iliac joint as well as anchors below the posterior superior iliac spines of the human body including an external shell having a length sufficient to extend around the abdominal region of the human body with fasteners at opposite ends of the shell and an air bladder disposed on the shell. The air bladder has a plurality of air chambers located centrally between opposite ends of the shell with each of the air chambers being in fluid communication with each other and to an air conduit for inflating the air chambers.

All of the previous inventions as described above have limitations in their ability to assist health care professionals in effectively treating lumbar spine conditions, as well as rehabilitating the upper and lower extremities. Thus it is seen that these previous efforts do not provide the benefits of a kinemometer stabilization program intended with the kit of the present invention or method, such as providing a portable and adaptable lumbar stabilization and upper and lower extremities rehabilitation muscle feedback kits to be used by the healthcare professional or the patient to measure changes in forces exerted upon the lumbar spine and upper and lower extremities.

The following example illustrates the novel and unique application of the lumbar stabilization component of the present invention. The spine stabilization muscle feedback device indicates the activity or non-activity of the lumbar spine stabilizers as they maintain or fail to maintain a “neutral spine” position. The activity or non-activity of the lumbar spine stabilizers is critical information required by healthcare professionals in determining proper muscle utilization or the ability of patients to maintain a “neutral spine”. Properly maintaining the “neutral spine” position indicates utilization of the lumbar spine stabilizers which is the basis for spine stabilization exercises. The present invention provides a measurable way for a healthcare professional or patient to determine proper muscle utilization or the ability of patients to maintain a “neutral spine”, thereby ensuring that the proper spine stabilizing muscle groups are being utilized to maintain a “neutral spine”, and consequently provides a device that would be easily adaptable and successfully used by healthcare professionals in the medical community. Additionally, prior techniques do not suggest the present inventive combination of component elements as disclosed herein.

Accordingly, it is seen that there is a need for a portable and adaptable lumbar stabilization and upper and lower extremities rehabilitation muscle feedback kinemometer stabilization program kit designed and configured to offer the most advanced technology available today to the medical community.

Thus, as will be seen, the present invention achieves its intended purposes, objectives and advantages over the prior art devices by accomplishing the needs and objectives as identified herein, through a new, useful and unobvious combination of component elements, which is simple to use, with the utilization of a minimum number of functioning parts, at a reasonable cost to manufacture, assemble, test and by employing only readily available material.

### SUMMARY OF THE INVENTION

The present invention is a novel and unique portable and adaptable lumbar stabilization and upper and lower extremities rehabilitation muscle feedback kinemometer stabilization program kit. The inventive devices and methods allow healthcare professionals as well as the patient to accurately assess the patient during the use of “neutral spine” exercises. The “neutral spine” exercises are used as effective treatment of lumbar spine conditions, as these exercises can be performed without actually engaging the muscles which stabilize the lumbar spine. Such engagement may hamper the positive effects of the “neutral spine” exercises. In addition, a portable and adaptable kinemometer stabilization program kit of the present invention also provides enhanced upper and lower extremities rehabilitation during the early strengthening phase of rehabilitation by improving patient isometric strength, as well as forcing the patient to maintain controlled contraction of the muscle, thereby increasing proprioception and neuromuscular control.

Utilizing the portable and adaptable kinemometer stabilization program kit of the present invention will innately enhance rehabilitation and reduce further injury to the lumbar spine, as well as the upper and lower extremities, thereby providing a means for quicker recovery which will consequently result in lower costs to hospitals and insurers.

A portable and adaptable kinemometer stabilization program kit of the present invention includes the following: complete kinemometer unit, spare kinemometer belt, instructional video, kinemometer calibrator (screw driver), instructional 8×11 cards and ordering brochures, potential warning for injury card, ball inflater, inflated balls having five, six and seven inch diameter for use with kinemometer exercises, kinemometer waterproof, shockproof container, instructions for use sheet.

In order to provide for such benefits, the kinemometer stabilization program kit of the present invention includes a complete kinemometer comprised of an elastic belt having a first end and a second end connected in the middle by a square shaped cloth envelope of material enclosing a rubber bladder. The second end is designed with one or two conventional “D” rings to receive and retain the first end. This will provide for a means of securing the elastic belt to the specific area of the patients body. In this way, the kinemometer is not reliant in all uses on capturing the pressurized bladder between the user's body and a external surface. Further, attached to the square shaped cloth of material enclosing the rubber bladder, by conventional tubing is a bladder valve. In addition, also attached to the square shaped cloth of material enclosing the rubber bladder is a second conventional tubing means comprising a unique and novel gauge located at the distal end, which is specifically designed for the purposes of the present invention. A lanyard is removably secured to the tubing directly below the gauge at a “T” interconnection of the gauge and the bladder valve.

During use of the kinemometer stabilization program kit of the present invention, the healthcare professional applies the elastic belt on the patients body in the desired position by placing hand through the lanyard and feeding the belt through the D rings and then adjusting the tension to the patients comfort level. The healthcare professional or patient then closes the bladder valve with thumb and four fingers clockwise and inflates to the desired firmness. At this stage, the patient would begin performing the indicated kinemometer stabilization program for treatment.

The present invention will allow the healthcare professional or the patient to effectively monitor the activity or non-activity of the lumbar spine stabilizing muscles during a treatment session utilizing “spine neutral” exercises. The instant invention provides an objective and measurable way for healthcare professionals or the patient to determine that the proper spine stabilizing muscles are actually engaged during the “spine neutral” treatment programs. In addition, during a treatment session for the rehabilitation phase for the neck, elbow or knee, the kinemometer stabilization program kit of the present invention provides improved isometric strength as a result of forcing the patient to maintain a controlled contraction of the neck, elbow or knee muscles during the use of the complete kinemometer unit. Also, during a treatment session for the early strengthening phase of rehabilitation of the upper and lower extremities, the kinemometer stabilization program kit of the present invention provides improved isometric strength as a result of forcing the patient to maintain a controlled contraction of the muscle during use of the complete kinemometer unit. Due to this muscle control, the muscle/joint must provide increased proprioceptive feedback which requires adequate neuromuscular control. Increased proprioception and neuromuscular control are not necessarily achieved through simple isometric-type strengthening exercises.

Using and operating the present invention is simple, effective and extremely efficient. The present invention will elevate the role of “spine neutral” and upper and lower extremity treatment programs in modern medicine. In addition, the novel and unique portable and adaptable lumbar stabilization and upper and lower extremities rehabilitation muscle feedback kit will greatly advance the technology in the art of “spine neutral”, as well as upper and lower extremities treatment programs.

Accordingly, it is the primary object of the present invention to provide for a lumbar stabilization and upper and lower extremities rehabilitation muscle feedback kinemometer stabilization program kit to be used by healthcare professionals or the patients with the capability to measure changes in forces exerted upon it, thereby proving healthcare professionals or the patients with an objective and measurable tool to determine activity or non-activity of the lumbar spine stabilizers which must be properly engaged during the use of “spine neutral” treatment programs for rehabilitating and preventing injury to the lumbar spine.

A further object of the present invention is to provide for a lumbar stabilization and upper and lower extremities rehabilitation muscle feedback kit which is completely portable and adaptable so as to increase versatility and functionality.

Yet another object of the present invention is to provide for a portable and adaptable kit which provides for a multipurpose rehabilitation program.

Still another object of the present invention is to provide for a lumbar elastic belt which is specifically shaped to enhance lumbar rehabilitation.

Yet another object of the present invention is to provide for a device which provides for the patient to participate vertically during the rehabilitation program.

Another object of the present invention is to provide for a lumbar stabilization and upper and lower extremities rehabilitation muscle feedback kinemometer stabilization program kit which is easily portable, adaptable, simple in design and one that can be utilized with a minimal amount of training.

A further object of the present invention is to provide for a portable and adaptable lumbar stabilization and upper and lower extremities rehabilitation muscle feedback kinemometer stabilization program kit that will overcome the deficiencies, shortcomings, and drawbacks of the prior devices used in the treatment of lumbar spine conditions.

A final object of the present invention to be specifically enumerated herein, is to provide a lumbar stabilization and upper and lower extremities rehabilitation muscle feedback kit in accordance with the preceding objects and which will conform to conventional forms of manufacture, be of simple construction and easy to use so as to provide a device that would be economically feasible, long lasting and relatively trouble free in operation.

The present invention meets the requirements of a simplified design, compact size, low initial cost, low operating cost, ease of installation and maintainability, and minimal amount of training to successfully employ the invention.

The foregoing has outlined some of the pertinent objects of the present invention. These objects should be construed to be merely illustrative of some of the more prominent features and application of the intended invention. Many other beneficial results can be obtained by applying the disclosed invention in a different manner by modifying the invention within the scope of the disclosure. Accordingly, a fuller understanding of the invention may be had by referring to the detailed description of the preferred embodiment, in addition to the scope of the invention taken in conjunction with the accompanying drawings.

### BRIEF DESCRIPTION OF THE DRAWINGS

FIG. 1 is a view of one embodiment of the present invention termed a kinemometer.

FIG. 2 is an enlarged view of the pressure gauge depicted in the embodiment of FIG. 1.

### DESCRIPTION OF THE PREFERRED EMBODIMENT

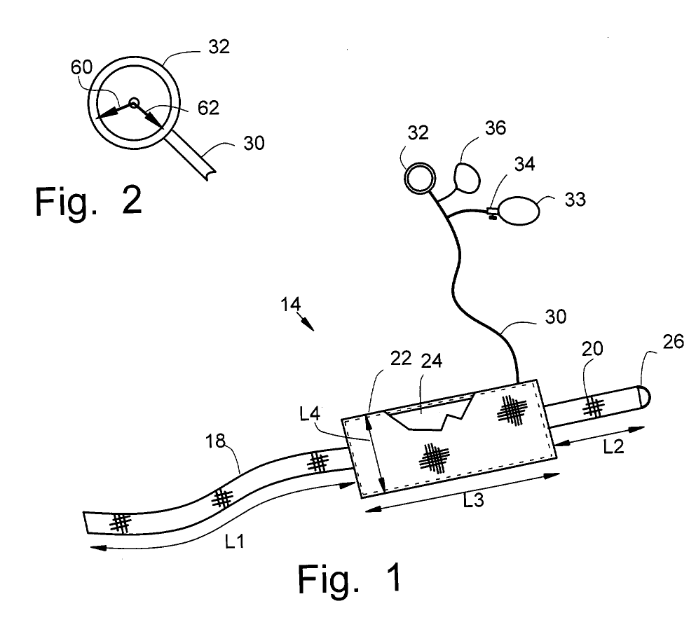

FIGS. 1 and 2 illustrate a preferred embodiment of the present invention. The physical embodiments of the invention including a bladder and pressure gauge are herein termed a “kinemometer”. The kinemometer in incorporated into a kinemometer stabilization program as a unique series of exercises designed to safely improve strength, endurance, coordination and proprioception of the trunk, lumbar and pelvic musculature. A kinemometer as herein described provides objective feedback to the exerciser and the clinician. It measures changes in forces exerted upon it. This indicates the activity (or lack of activity) of lumbar spine stabilizers as they maintain, or fail to maintain, a “neutral spine” position.

The kinemometer 14 of the present invention is comprised of an belt having a first belt end 18 and a second belt end 20 connected in the middle by a square shaped cloth envelope 22 of material enclosing a rubber bladder 24. The belt is preferably formed of a sturdy elastic material. The envelope 22 may be formed of the same material or other durable fabric or plastic. The second end 20 is designed with a conventional “D” ring fastener, or the like, to adjustably receive and retain the first end 18. The function of the belt and D ring is to secure the envelope with the bladder to a human body part such as a leg, neck, or lumbar spine region.

FIG. 1 defines dimensions for a most preferred embodiment that enables a broad range of applications based on human body measurements. The first belt end 18 has a length L1 of 25 inches. The second belt end 20 has a length L2 of 11 inches. The envelope 22 has an effective internal dimensions L3 and L4 of 16 inches and 8 inches, respectively. These effective dimensions L3 and L4 are designed to accept a bladder of the same size.

Attached to the bladder 24 and passing out of the envelope 22 is a pressure tube 30 in communication with the internal volume of the bladder 24. The tube is preferably rubber or such material as is typically used for low pressure gas transfer.

Connected to the distal end of the tube 30 is a pressure gauge 32 and manual hand pump 33 with a bladder valve 34. The hand pump 33 enables pressurization of the bladder 24 through the tube 30 when the bladder valve 34 is in a closed position. The bladder valve 34 also enables release of pressure from the bladder 24. The design and operation of the bladder valve and hand pump, individually, are well known to those skilled in the art. In the figure, the hand pump 33 and bladder valve are shown connected at a “T” joint in the tube 30. Alternatively, these elements may be connected to the bladder through a separate second tube. The tube preferably has a length of 48 inches from the bladder to the gauge. A lanyard 36 is removably secured to the tube 30 directly below the gauge. The lanyard is sized and configured to comfortably encircle a user's hand. The function of the lanyard 36 is to enable easy manipulation and retention of the gauge 32 by the user during exercising as part of the inventive methods.

The invention includes a kit having one or more kinemometers in a protective case. Kits of the present invention may also include the following items: complete kinemometer 14, spare kinemometer belt, instructional video, kinemometer calibrator (screw driver), instructional cards, ball inflater, and a five, six and seven inch diameter inflatable ball, and written instruction sheets.

During inventive methods of use of the kinemometer, the patient user places their hand through the lanyard 36, places the belt around their waist to feed the first end of the belt 18 through the D rings 26 located at the second end of the belt 20. The patient may adjust the tension of the elastic belt 16 to their comfort level by pulling the first end of the belt 18 further through the D rings 26 for a tighter fit, or by feeding less of the first end of the belt 18 through the D rings 26 for a looser fit. Then the patient closes the bladder valve 34 with the thumb and four fingers clockwise and inflates bladder 24 using the manual hand pump 33. The combination of the restraint of the belt and envelope, and inflation pressure of the bladder ensures close communication of the bladder with the portion of the user's body contacting the envelope. In this manner, muscular tension and body structural changes are reflected in the bladder pressure. With the manual hand pump 56 inflated to the appropriate firmness, the patient will begin the kinemometer stabilization program exercise as designated in the methods of use.

The gauge 32 is shown more clearly in FIG. 2. Preferably, the gauge of the present invention is especially large for ease of use and increased visibility. The gauge is preferably comprised of a 3.5 inch diameter body featuring a beryllium copper diaphragm with a brass socket. The gauge should have an useful pressure range of 0 to 100 as typically measured by inches of water. The gauge preferably also has a nonactive first dial hand 60 and an active second dial hand 62. The first dial hand 60 allows a set pressure level to be statically indicated on the dial. The active dial hand 62 indicates the current pressure. During use, the position of the active dial hand 62 may be easily compared to the position of the nonactive dial hand 60 to determine changes in bladder pressure. A kit according to the present invention includes a calibrator formed as a screwdriver which provides for quick and easy means for adjusting the nonactive dial hand. Prior to a program exercise or the like, the nonactive dial hand is adjusted to a predetermined “zero zone” or target pressure. While wearing the kinemometer unit of the present invention, in between exercises, the gauge may be stowed between the patients body and the elastic belt.

Additionally, the present invention provides for kinemometer exercise variations, thus providing a valuable tool in reducing recovery time in the rehabilitation of the upper and lower extremities. Through the use of the kinemometer stabilization program kit, the patient will improve isometric strength, as well as increase proprioception and neuromuscular control.

In an alternative embodiment, the present invention provides for an alternative version of the kinemometer stabilization program kit. This kit is comprised of a complete kinemometer 14, instructional sheets, warning for potential injury sheet, calibrator (screwdriver), and a waterproof and shockproof container.

While the present invention has been particularly shown and described with reference to a specific embodiment thereof, it will be understood by those skilled in the art that various changes in form and detail may be made without departing from the spirit and scope of the present invention.

I claim:

1. A muscle rehabilitation device comprising:
a bladder configured to maintain internal pressure;
an envelope surrounding the bladder;
an adjustable belt configured to encompass a human body portion and secured to the envelope;
a viewable pressure monitor connected to the bladder; and
a hand pump for pressurizing the bladder.

2. The device of claim 1, further comprising:
a lanyard secured to the gauge.

3. The device of claim 1, wherein:
the pressure monitor is a gauge having a viewable nonactive dial hand and an active dial hand.

4. 1. A muscle rehabilitation device comprising:
an inflatable bladder;
portable means of retaining the bladder against a human body portion;
a viewable pressure monitor connected to the bladder and having a means of indicating a preset pressure level; and
a hand pump for pressurizing the bladder.

5. A method of muscle rehabilitation comprising the steps of:
retaining a bladder against a body portion while enabling the body portion to be displaced;
pressurizing the bladder;
establishing an initial bladder pressure; manipulating the body while monitoring the bladder pressure to maintain a constant pressure;

6. The method of claim 5, further comprising:
setting a viewable pressure indicator to the initial bladder pressure.
